(19) United States
(12) Patent Application Publication
(10) Pub. No.: US 2006/0009684 A1
(43) Pub. Date: Jan. 12, 2006

(54) System for monitoring compliance to a healthcare regiment of testing

(76) Inventor: Steven Kim, Harrington Park, NJ (US)

Correspondence Address: Donald L. Monin, Jr.;JENKENS & GILCHRIST, A PROFESSIONAL CORPORATION, Suite 900, 901 15th Street, Washington, DC, 20005, US

(21) Appl. No.: 11/175,382

(22) Filed: Jul. 7, 2005

Related U.S. Application Data
(60) Provisional application No. 60/586,132, filed on Jul. 7, 2004.

(51) Int. Cl. A61B 5/00

(52) U.S. Cl. 600/300

(57) ABSTRACT

A system for monitoring compliance to a health care regiment of testing includes a wireless communication device for sending and receiving communication signals, at least one bio-sensor within the wireless communication device for providing a medical test of an individual in accordance with the health care regiment, a memory within the wireless communication device for recording test results from the medical test, a carrier network for receiving data from the wireless communication device that includes test results, and a service provider for receiving the data via the carrier network, the service provider analyzing the test results in view of the healthcare regiment to determine if a message should be sent by the service provider to a receiving device of at least one of a mentor and a healthcare professional for the individual.

The present invention claims the benefit of Provisional Application Ser. No. 60/586,132 filed on Jul. 7, 2004, which is hereby incorporated by reference in its entirety.

### BACKGROUND OF THE INVENTION

1. Field of the Invention

The present invention relates to a monitoring system, and more particularly, to a system for monitoring compliance to a health care regiment of testing. Although the present invention is suitable for a wide scope of applications, it is particularly suitable for testing and monitoring a chronic disease.

2. Discussion of the Related Art

Today, a large portion of the population is coping with a chronic disease, such as diabetes, liver disease or heart disease. Treatment for such diseases not only include medicines but can also include changes in lifestyles. For example, a change in diet and/or exercise routines may be advised. Further, constant medical testing may be required.

There are several chronic diseases that not only require a change in lifestyle but also require medical testing. Of course, most people desire that such testing or monitoring be as non-invasive as possible. Further, people do not want to spend time generating reports or providing other types of medical testing data to their doctor. Meanwhile, doctors prefer to have a consistent and constant stream of medical testing data. More particularly, doctors like to see clear indications that a prescribed regiment is being pursued and to be notified as quickly as possible if there are any problems. Further, people with chronic diseases are more likely to need intervention or some type of emergency assistance if the monitoring data indicates that there is a problem.

Various medical apparatuses have been proposed to monitor individuals. Lately, apparatuses have designed such that the individuals can monitor themselves. For example, U.S. Pat. No. 6,558,320 discloses a personal data assistant that can be directly coupled to a medical device or wirelessly connected to a medical device to measure blood glucose levels of a diabetic. Such a device reliable stores data for each monitoring and can track trends based on the data of each monitor. Such information is of little use unless it is viewed and analyzed by a healthcare professional.

Numerous systems have been suggested for the data management of patient information such that a healthcare professional can diagnose or prescribe treatments for individuals. For example, U.S. patent application Publication US 2004/0073464 discloses a method and systems for data management in patient diagnoses and treatment using a fax machine. However, the system of U.S. patent application Publication US 2004/0073464 depends on the patient to provide a download to a central system.

Typically, a monitored person has to carry the monitoring equipment as well as other personal items. For example, a diabetic may carry a glucose meter in addition to a cell phone or other types of wireless communication devices, such as a pager. Carrying multiple items can be cumbersome and inconvenient.

### SUMMARY OF THE INVENTION

Accordingly, the present invention is directed to a system for monitoring compliance to a healthcare regiment that substantially obviates one or more of the problems due to limitations and disadvantages of the related art.

An object of the present invention is to provide a system that allows an individual to take a medical test and automatically provides a report regarding the medical test to at least one of a healthcare professional and a mentor.

Another object of the present invention is to provide a system that monitors both exercises performed by the individual and a medical test taken by the individual.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, a system for monitoring compliance to a health care regiment of testing includes a wireless communication device for sending and receiving communication signals, at least one bio-sensor within the wireless communication device for providing a medical test of an individual in accordance with the health care regiment, a memory within the wireless communication device for recording test results from the medical test, a carrier network for receiving data from the wireless communication device that includes test results, and a service provider for receiving the data via the carrier network, the service provider analyzing the test results in view of the healthcare regiment to determine if a message should be sent by the service provider to a receiving device of at least one of a mentor and a healthcare professional for the individual.

In another aspect, a system for monitoring compliance to a healthcare regiment of testing includes a first cell phone for telephonic communications, at least one bio-sensor within the cell phone for providing a medical test of an individual in accordance with the healthcare regiment, a memory for recording test results from the medical test and other information input into the cell phone by the individual, a carrier network for receiving data from the cell phone that includes the test results and the other information, and a service provider for receiving the data via the carrier network, wherein the cell phone is programmed to send a message via the carrier network after a medical test is taken to provide the results of the medical test to a second cell phone.

In another aspect, a system for monitoring compliance to a healthcare regiment of testing includes a cell phone for sending and receiving communication signals, a glucose meter for providing a medical test of an individual in accordance with the healthcare regiment, a memory for recording test results from the medical test and other information input into the wireless communication device by the individual, a carrier network for receiving data from the cell phone that includes the test results and the other information, and a service provider for receiving the data via the carrier network, the service provider analyzing the test results in view of the healthcare regiment and sending a message to a receiving device of at least one of a mentor and a healthcare professional for the individual.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 is a diagram of a system for monitoring compliance to a health care regiment of testing in accordance with an embodiment of the present invention.

FIG. 2 is an example of a display screen on a cell phone for a blood sugar test result taken by a glucose meter built into the cell phone.

FIG. 3 is an example of a display screen on a cell phone having a record inquiry for input of additional information.

FIGS. 4A-4C are examples of drop down menus used for the input of additional information.

FIG. 5 is an example of a display screen on a cell phone for an activity report received by either a mentor or a healthcare professional.

### DETAILED DESCRIPTION OF THE PREFERRED EMBODIMENTS

Reference will now be made in detail to the preferred embodiments of the present invention, examples of which are illustrated in the accompanying drawings.

One of the most critical factors in determining whether an individual will have long term success in dealing with a chronic disease, such as diabetes, is whether the individual will maintain compliance with a healthcare regiment of testing as well as a healthy lifestyle. Some people need reminders for testing times, while other people need positive reinforcement or direct monitoring to make sure that the testing is performed. For example, teenage diabetics sometimes try to skip glucose testing or forget to perform a glucose test. Since most people constantly carry the cellular phones, cell phones appear to be an ideal vehicle for applying mobile healthcare assistance.

A cell phone has been developed to include bio-sensors within the cell phone. More particularly, the bio-sensors of the cell phone include a glucose-meter and a pedometer that are actually within the cell phone. Such a cell phone enables (1) blood sugar monitoring using built-in glucose meter, (2) exercise monitoring using built-in pedometer, (3) food log-in either by manual entry or by capturing a picture using built-in camera. Such a phone can be used in a web/cell phone based system managed by healthcare professionals to monitor the compliance of individual to a healthcare regiment of testing.

A phone cell phone with built-in bio-sensor, such as a glucose meter, can be used as a part of system to notify a mentor for an individual. A parent, guardian, relative, healthcare professional or a friend can be a mentor for an individual. The mentor assists or inspires the individual to comply with a healthcare regiment of testing. Such a system is especially effective for use with the adolescent population. Further, such a system can be used to monitor an individual in an effort to prevent unattended long-term unconsciousness of the individual due to hypoglycemia. Furthermore, such a system can be used to automatically provide blood sugar testing results to a service provider that can record the results. The tracking of such test results is very important for individuals with Type I diabetes.

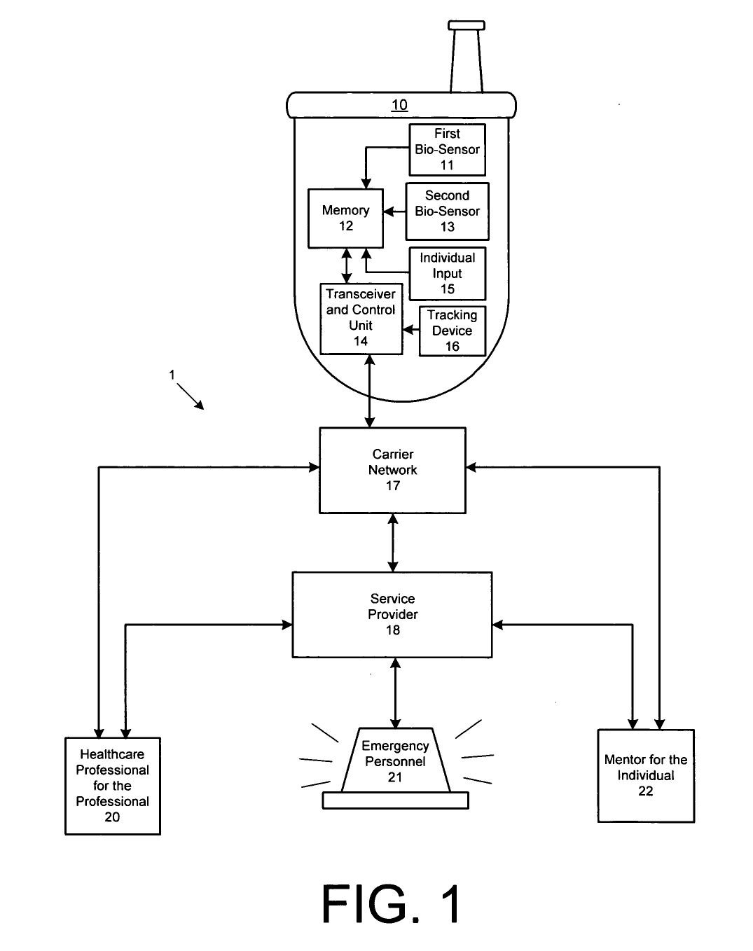

FIG. 1 is a diagram of a system for monitoring compliance to a health care regiment of testing in accordance with an embodiment of the present invention. The system includes a wireless communication device, such as a cell phone 10, that communicates with a service provider 18 through a carrier network 17. The service provider 18 can send and receive communications from a receiving/sending device 20 of a mentor for the individual, a receiving/sending device 21 of emergency personal or a receiving/sending device 22 of a healthcare professional for the individual.

The mentor for an individual can be a guardian, parent, friend or relative. A healthcare professional for the individual can be the individual's personal doctor, another doctor, a nurse or a clinician. The emergency personnel can be people in an emergency room, emergency clinic or other types of emergency response people, such as paramedics. For example, the emergency personnel may be the local emergency rescue service.

The wireless communication device can be a cell phone 10 having a first bio-sensor 11, such as a glucose meter. The first bio-sensor 11 can be used by an individual to perform a medical test, such as a blood sugar test. The results of the test are stored in the memory 12 of the cell phone 10. The cell phone 10 can also include a second bio-sensor 13, such as pedometer, that takes measurements of physical activity, which can be stored in the memory 12 of the cell phone 10. Further, the cell phone 10 has an individual input 15 capability such that an individual can enter additional information into the memory 12. The cell phone 10 has a transceiver and control unit 14 that controls telephonic communications and the sending of data to the service provider 18 via the carrier network 17. The cell phone 10 can have a tracking device 16, such as a geographical positioning sensor (GPS) 16, for locating the cell phone 10. The transceiver and control unit 14 also controls the flow of positional information from the tracking device 16 to the service provider 18 via the carrier network 17. Further, the transceiver and control unit 14 also responds to data queries received from the service provider 18 via the carrier network 17.

The data that is sent to the service provider can include the test results of the first bio-sensor 11, measurements of the second bio-sensor 12, and additional information input into the memory 12 by the individual as prompted by the cell phone. Further, the data can also include information regarding incoming and outgoing calls that is stored in the memory 12. Other data input by the individual can also be input into the memory 12.

Although a cell phone 10 is depicted as the wireless communication device in FIG. 1, the wireless communication device can also be a wireless capable personal data assistant or pager in which the glucose meter is built-in. The cell phone 10 can include a camera as well as a media player, such as an MP3 player. The cell phone 10 can also have the capabilities of a personal data assistant and/or a pager.

The blood sugar test can be the invasive type in which a testing strip is inserted into a slot of the glucose meter built into the cell phone. The insertion of the strip into the cell phone initiates the blood sugar test procedure. In the alternative, the first bio-sensor can be a non-invasive type of glucose meter.

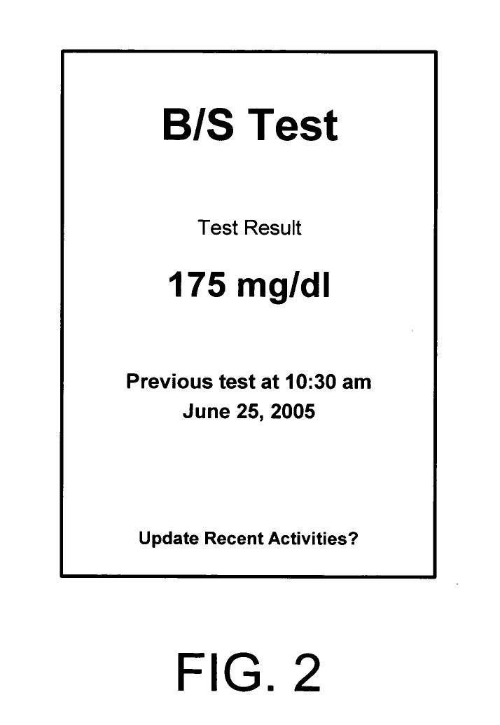

FIG. 2 is an example of blood sugar test results on a display screen of a cell phone for a blood sugar test performed by a glucose meter built into the cell phone. After the glucose measurement is taken, a one touch button on the cell phone will send the blood sugar test results to the service provider 18. In case the data has not been sent from the cell phone 10 due the failure to communicate with carrier network, the data will be stored in the memory 12 until it is later down-loaded (synchronized) to the service provider 18 either automatically or manually.

The cell phone can also send a brief short message service (SMS) message of the blood sugar testing result to designated phone numbers of cell phones on the carrier network 17, such as the mentor's cell phone. The SMS message can include testing time and blood sugar level. Further, the mentor can send an SMS message back. In addition, the cell phone can send an SMS message to the mentor if a blood test is not performed within a predetermined period. The cell phone can configured such that an SMS message is not sent when a blood test is taken and a message is only sent to the mentor if the blood test is not performed within a predetermined period.

The cell phone 10 with a built-in glucose meter can be programmed with a blood sugar test schedule in accordance with a healthcare regiment for the individual. The cell phone 10 can remind the individual of the need for a test with an alarm. Further, the cell phone 10 can also include a built-in pedometer that continuously monitors calories burned, distance traveled, and the time the individual actually spent on the activity that is burning the calories. Like the blood sugar test results, the recent pedometer data can also be automatically or manually down-loaded to the service provider 18.

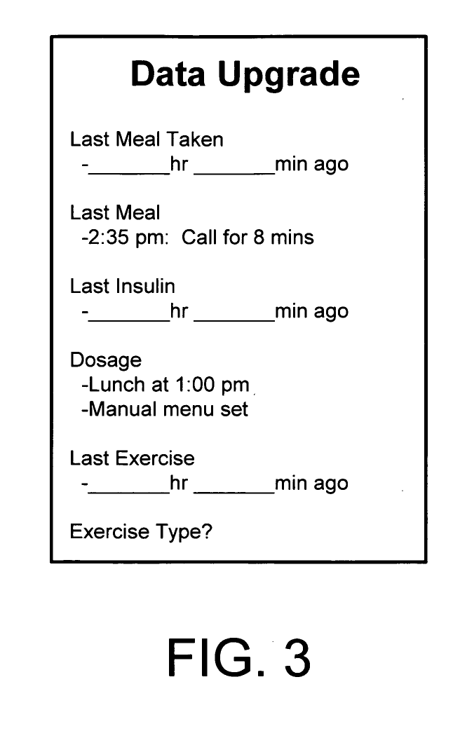

The cell phone 10 with a built-in glucose meter also has software that prompts for additional information after a blood sugar measurement. FIG. 3 is an example of a display screen on a cell phone having record inquiry for input of additional information. A menu driven record inquiry can be used to input the additional information. The software can also be used to describe the activity measured by the pedometer.

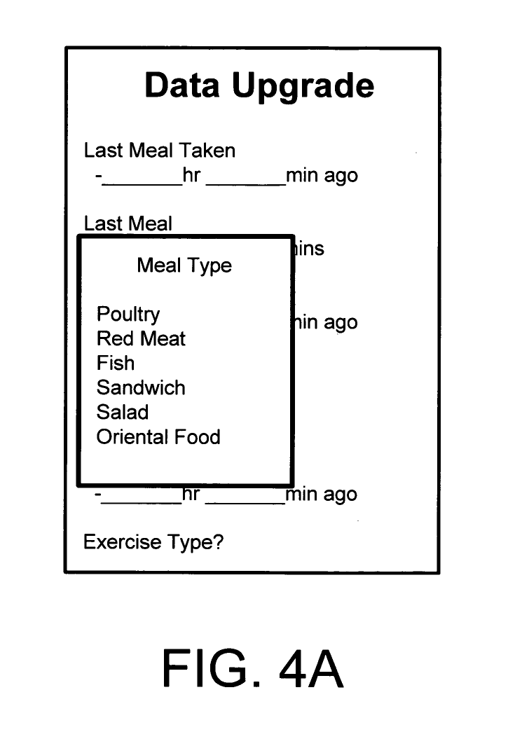

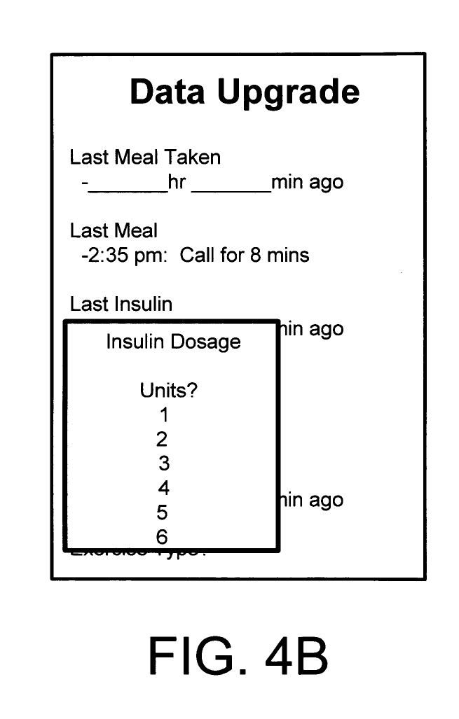

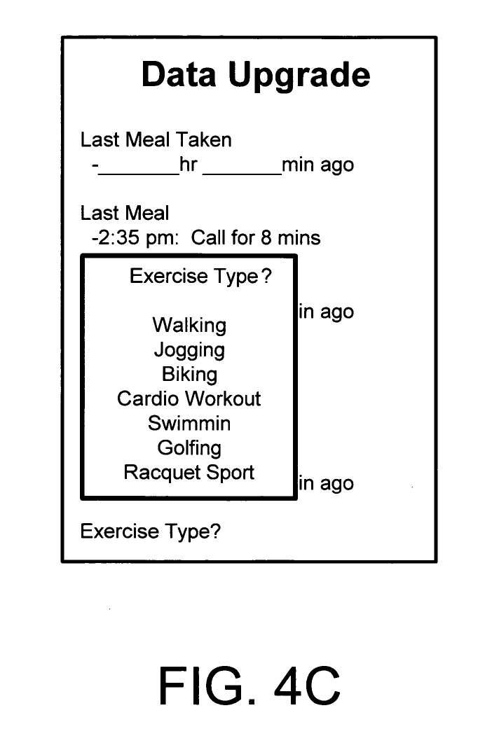

FIGS. 4A-4C are examples of drop down menus used for the input of additional information. The additional information can also be used with regard to the diet of the individual. After each blood sugar testing, the cell phone displays a screen inquiring about the recent meal, medication and exercise. The entries for the inquiries can be entered using a drop down menu format. One can easily enter the meals has taken recently. For example, a drop down food list with automatic carbohydrate conversion can be provided. Further, the cell phone can be provided with a camera and a snap-shot of a meal can be taken, so that it can be sent to the service provider 18. A special optional service can be offered in which an analysis of the food is done by a dietician based on the snap-shot of the meal. Thus, the cell phone 10 can provide easy record keeping.

The carrier network 17 can be a cellular communications network that can also handle data transfers between the service provider 18 and the cell phone 10. The wireless communication device is programmed such that data can be sent to the service provider 18, via the network carrier 17. Further, the carrier network 17 can be capable of multimedia messaging service (MMS) for the transfer of graphic, audio or video files between the service provider 18 and the cell phone 10. The use of MMS is a user-friendly, effective and efficient intervention tool, especially for diabetes management of the adolescent population.

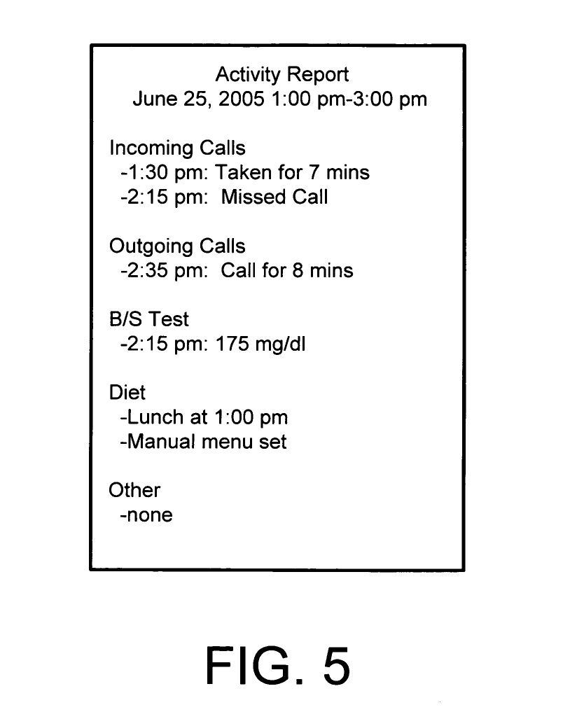

The cell phone 10 can further be programmed to send a message through the short message service (SMS) of the carrier network 17 after a medical test is taken to show the results of the medical test to the receiving/sending device 20 of a mentor for the individual. In the alternative, the cell phone 10 can also be programmed to send a test result message through the short message service (SMS) of the carrier network 17 after a medical test is taken to show the results of the medical test in an activity report to one of or both the receiving/sending device 20 of a mentor for the individual and the receiving/sending device 22 of a healthcare professional for the individual. FIG. 5 is an example of a display screen on a cell phone for an activity report received by either a mentor or a healthcare professional. The wireless communication device can be configured such that the test result message is sent for every medical test reading done by the first bio-sensor 11 or only for a medical test result that is abnormal, such as a blood sugar measurement that is too high or too low.

The service provider 18 is, for example, a computer that is web/cell phone accessible. The service provider 18 can also include operators overseeing operations of the computer. The cell phone 10 can be configured to download the data recorded in the memory 12 to the service provider 18 periodically and automatically. Such a configuration not only ensures data synchronization but more importantly, an individual's activities can be checked by others for indications that the individual may be or may soon become unconscious. The data can include records such as the last time in incoming phone call was taken, the last time an outgoing call was made, a first bio-sensor reading, a second bio-sensor reading, other data recorded by the individual and the location of the wireless communication device. Such data is stored at the service provider 18 in a personal database for the individual. In addition or in the alternative to the service provider 18 automatically receiving data from the wireless communication device 19, the service provider 18 can periodically query the cell phone 10 for the latest data in the memory 12 of the communication device.

The service provider 18 can be configured to send an automatic periodic message to the receiving/sending devices of mentors and healthcare professionals for the individual. In a situation in which no activities have occurred over a period of time or within a certain period of time, which can be defined or configured by the individual, the individual's guardian or the individual's physician, an automatic emergency message can be sent to the receiving/sending device 21 of emergency personnel. The automatic emergency message can include medical information, such as diabetes related information, and the last known location of the individual as indicated by the tracking device 17 in the cell phone 10. The data in the service provider 18 can also be accessed with a proper identification and password by the mentors and healthcare professionals for the individual as well as emergency personnel at anytime. Further, the service provider can provide weekly activity reports of the data to the individual's physician.

As described above, the cell phone 10 can be pre-programmed into the cell phone 10 so as to provide easy record keeping of the medical test. Further, such a plan can also be pre-programmed into the service provider 18. Thus, the service provider 18 can provide alert the mentor of an insulin/drug time for the individual.

The service provider 18 can be accessed by the individual, the mentor, the healthcare professional for the individual or emergency personnel. After accessing the service provider 18 with a proper identification and password, the parameters for an emergency can be set. For example, no activity within a set amount of hours or no activity within a set period can be defined as an emergency. In addition or in the alternative, an emergency can be a blood sugar level that is too high or too low with respect to a personal blood sugar level. The levels of what is too high or too low are stored in the service provider 18 and can also be stored in the cell phone 10. Other information can also be provided to the service provider 18 for use in an emergency, such as the contact information for the individual's physician as well as other emergency contact information.

The service provider 18 can be programmed to take a series of steps in response to an emergency situation being detected. For example, when the measured blood sugar level is too high or too low, automatic emergency messages can be sent to a mentor's cellular phone by either SMS or voice call as well as to emergency personnel. The emergency message can include recent activity information such as past the results of past medical tests and the last known position of the individual based on the tracking device in the wireless communication device. The emergency message can be sent in series to one or more people until the message is accepted. Further, the emergency message can be continuously sent to a group of people until one of the people in the group accepts the message. The service provider 18 can continuously send messages to a group of mentors until the message is accepted while also sending messages to a group of healthcare professionals as well as to the emergency personnel.

The receiving/sending devices can be a personal data assistant, a blackberry, a cellular phone or a personal computer. The receiving/sending device can also be a fax machine, telephone or pager for receiving messages from the service provider 18. Because the service provider is a computer, data entry devices, such as a personal data assistant, a blackberry, a cellular phone or a personal computer, are preferably the receiving/sending device.

It will be apparent to those skilled in the art that various modifications and variations can be made in the a system for monitoring compliance to a health care regiment of testing and monitoring of the present invention without departing from the spirit or scope of the invention. Thus, it is intended that the present invention cover the modifications and variations of this invention provided they come within the scope of the appended claims and their equivalents.

What is claimed is:

1. A system for monitoring compliance to a health care regiment of testing, comprising:
a wireless communication device for sending and receiving communication signals;
at least one bio-sensor within the wireless communication device for providing a medical test of an individual in accordance with the health care regiment;
a memory within the wireless communication device for recording test results from the medical test;
a carrier network for receiving data from the wireless communication device that includes test results; and
a service provider for receiving the data via the carrier network, the service provider analyzing the test results in view of the healthcare regiment to determine if a message should be sent by the service provider to a receiving device of at least one of a mentor and a healthcare professional for the individual.

2. The system of claim 1, wherein the wireless communication device is a cell phone.

3. The system of claim 1, wherein the receiving device is one of a cell phone, personal computer and fax machine.

4. The system of claim 1, wherein the bio-sensor is a glucose meter.

5. The system of claim 1, wherein the wireless communication device includes a second bio-sensor.

6. The system of claim 5, wherein the second bio-sensor is a pedometer for measuring physical activity.

7. The system of claim 6, wherein the data includes measurements from the pedometer.

8. The system according to claim 1, wherein the memory receives other information that is input into the wireless communication device by the individual.

9. The system of claim 8, wherein the data includes the other information input into the wireless communication device by the individual.

10. The system of claim 9, wherein the other information describes a dietary intake of the individual.

11. The system of claim 10, wherein the wireless communication device includes a camera and the other information is snap-shot of a meal.

12. The system of claim 1, wherein the wireless communication device is programmed to send a message via the carrier network after a medical test is taken to show the results of the medical test to a receiving device of at least one of the mentor and the healthcare professional for the individual.

13. The system of claim 1, wherein the service provider sends a message to at least one of the mentor and the healthcare professional for the individual if no medical test data is received for a particular predetermined time period after a predetermined amount of time.

14. The system of claim 1, wherein the service provider periodically queries the wireless communication device for data of a predetermined period.

15. The system of claim 1, wherein the service provider sends a message to at least one of the mentor and the healthcare professional for the individual if no medical test data is received for a particular predetermined time period after a predetermined amount of time.

16. A system for monitoring compliance to a healthcare regiment of testing, comprising:
a first cell phone for telephonic communications;
at least one bio-sensor within the cell phone for providing a medical test of an individual in accordance with the healthcare regiment;
a memory for recording test results from the medical test and other information input into the cell phone by the individual;
a carrier network for receiving data from the cell phone that includes the test results and the other information; and
a service provider for receiving the data via the carrier network,
wherein the cell phone is programmed to send a message via the carrier network after a medical test is taken to provide the results of the medical test to a second cell phone.

17. The system of claim 16, wherein the bio-sensor is a glucose meter.

18. The system of claim 16, wherein the cell phone includes a pedometer for measuring physical activity.

19. The system of claim 18, wherein the data includes measurements from the pedometer.

20. The system according to claim 19, wherein the other information describes the activity measured by the pedometer.

21. The system of claim 16, wherein the other information describes the dietary intake of the individual.

22. The system of claim 16, wherein the first cell phone includes a camera and the other information is snap-shot of a meal.

23. The system of claim 16, wherein the service provider sends a message to at least one of a mentor and a healthcare professional for the individual if no medical test data is received for a particular predetermined time period after a predetermined amount of time.

24. The system of claim 16, wherein the service provider periodically queries the wireless communication device for data of a predetermined period.

25. The system of claim 16, wherein the service provider sends a message to at least one of a mentor and a healthcare professional for the individual if no medical test data is received for a particular predetermined time period after a predetermined amount of time.

26. A system for monitoring compliance to a healthcare regiment of testing, comprising:
a cell phone for sending and receiving communication signals;
a glucose meter for providing a medical test of an individual in accordance with the healthcare regiment;
a memory for recording test results from the medical test and other information input into the wireless communication device by the individual;
a carrier network for receiving data from the cell phone that includes the test results and the other information; and
a service provider for receiving the data via the carrier network, the service provider analyzing the test results in view of the healthcare regiment and sending a message to a receiving device of at least one of a mentor and a healthcare professional for the individual.

27. The system of claim 26, wherein the receiving device is one of a cell phone, personal computer and fax machine.

28. The system of claim 26, wherein the other information describes a dietary intake of the individual.

29. The system of claim 26 wherein the wherein the cell phone includes a bio-sensor.

30. The system of claim 29, wherein the bio-sensor is a pedometer for measuring physical activity.

31. The system of claim 30, wherein the data includes measurements from the pedometer.

32. The system according to claim 26, wherein the other information describes the activity measured by the pedometer.

33. The system of claim 26, wherein the cell phone automatically sends the data to the service provider after a medical test is taken and then the service provider sends the test results to one of the mentor and the healthcare professional for the individual.

34. The system of claim 26, wherein the service provider sends a message to at least one of the mentor and the healthcare professional for the individual if no medical test data is received for a particular predetermined time period after a predetermined amount of time.

35. The system of claim 26, wherein the service provider periodically queries the wireless communication device for data of a predetermined period.

36. The system of claim 26, wherein the service provider sends a message to at least one of the mentor and the healthcare professional for the individual if no medical test data is received for a particular predetermined time period after a predetermined amount of time.
